(19) United States
(12) Patent Application Publication
(10) Pub. No.: US 2010/0285098 A1
(43) Pub. Date: Nov. 11, 2010

(54) ADHERING TROCHES WITH SANTACID FOR TREATMENT OF THROAT ESOPHAGUS AND STOMACH

(76) Inventor: Jeffrey T. Haley, Bellevue, WA (US)

Correspondence Address: Jeffrey T Haley, 13434 SE 27th Place, Bellevue, WA, 98005, US

(21) Appl. No.: 12/735,017

(22) PCT Filed: Sep. 10, 2008
(86) PCT No.: PCT/US2008/075885

§ 371 Date: Jun. 11, 2010

Related U.S. Application Data
(63) Continuation-in-part of application No. 61/063,061, filed on Jan. 30, 2008.
(60) Provisional application No. 60/993,457, filed on Sep. 11, 2007.
(60) Provisional application No. 60/995,754, filed on Sep. 27, 2007.

(51) Int. Cl. A61K 9/34; A61K 33/10; A61K 33/08; A61K 33/00

(52) U.S. Cl. 424/435; 424/686; 424/687; 424/690; 424/692; 424/693; 424/715; 424/717; 424/722

(57) ABSTRACT

A method for combating unwanted conditions downstream of the mouth with adhering troches. A method to treat or reduce replication of a virus in throat tissues by releasing an anti-viral from an adhering disc. A method to treat sore throat with time release of Glycyrrhiza extract or collagen from an adhering disc. A method to treat sore throat with topically applied cobalamin. An adhering troche that releases antacid, alginate, bismuth subsalicylate, soluble zinc, such as zinc gluconate, bioactive vitamin B12 (methylcobalamin), antibiotic, or anti-viral. A method to combat bacterial effects downstream of the mouth with topically applied xylitol.

### BACKGROUND

Diseases of the throat, esophagus, and stomach have long been treated with topically active ingredients either swallowed as a liquid or chewed and swallowed as a slurry. In each case, the topical treatment is brief as the liquid or slurry passes by. Such materials do not rest long even in the stomach which passes them to the duodenum. Except when it is processing food, which dilutes any topical medication, swallowed items that enter the stomach do not linger. For this reason, medications which act topically on the throat, esophagus, or stomach must be frequently ingested to remain effective.

“Peptic ulcer disease” consists of ulcers in the upper gastrointestinal tract, the esophagus, stomach and duodenum, from various causes, mostly infection with Helicobacter pylori bacteria. Topical medications for treatment include: (1) Mucosal protective agents which protect the stomach's mucous lining from acid, such as alginate (Gaviston®), sucralfate (Sulcrate® or Carafate®) and misoprostol (Cytotec®), (2) antacids, such as sodium bicarbonate (Alka Seltzer®), sodium carbonate, calcium carbonate and others (Tums® and Rolaids®), and (3) bismuth subsalicylate (Pepto-Bismol® and Kaopectate).

Other medications for treatment of peptic ulcers, such as H2 blockers, proton pump inhibitors, and antibiotics (such as metronidazole (Flagyl®), tetracycline (Achromycin® or Sumycie), amoxicillin (Amoxil® or Trimox®), and clarithromycin (Biaxin®) are taken by mouth as swallowed pills or liquids, but their topical effect is insignificant. Instead, they achieve an effect longer than the transit time of the stomach by passing from the gut to the blood and then to the site of action, requiring that they achieve a high concentration throughout the body tissues even though their action is only needed in a very small portion of the body tissues.

A related painful condition of the esophagus, gastroesophageal reflux disease or acid reflux, is caused by stomach acids rising into the esophagus. If untreated, it can cause an ulcer of the esophagus, which is within the class of peptic ulcers, and can erode teeth. Topical medications for treatment are a subset of the above medications for peptic ulcer: (1) mucosal protective agents which protect the throat's mucous lining from acid, and (2) antacids. Because there is no delivery of the medications over time, the medications may be taken at a time of day just before the problem typically manifests. Typically, they are taken in response to a symptom for immediate effect and then taken again as often as needed because the effect is short acting due to transit of the medication.

Sore throat (pharyngitis) may be caused by bacteria, such as Streptococcus pyogenes, or viruses, such as cold or flu viruses, or by irritants in air or consumed food or drink. If bacterial, the best treatment may be systemic antibiotics. Some viral infections may respond to systemic anti-virals. Otherwise, the best treatments are topical such as from throat lozenges or “cough drops”.

Picornavirus is a family of viruses that includes many genera that infect the throat and nearby surface tissues, including rhinoviruses, usually called “cold” viruses. Influenza viruses also infect the throat and nearby surface tissues. The prevailing view is that all of these viruses first lodge in the pharynx or nearby tissues where they first replicate and then spread to adjoining mucous tissues including the lungs and sinuses. There are two ways to deliver drugs effective against viruses that replicate in these tissues: by topical delivery or by delivery through the blood. Methods for topical delivery include nasal spray and fine powder inhaler for topical delivery into the lungs and airways.

Oral care researchers have established that frequent delivery of xylitol molecules in the mouth can reduce plaque, caries, and inner ear infections by suppressing the growth of certain bacteria. These bacteria thrive on certain carbohydrate molecules such as sucrose, glucose, fructose and other sugars but, when they ingest the xylitol molecules, they cease proliferating and cease to adhere to human tissues. Helicobacter pylori, which causes about 80% of peptic ulcers, and Streptococcus pyogenes, which causes “strep throat,” are both suppressed by xylitol.

Mints, lozenges, and lollipops may be technically described as “troches”. For treatment of health problems in the mouth or throat, people have for centuries held in their mouths a composition containing medication for topical application. Since the middle ages, the name for such a composition, derived from Latin and previously from Greek, is “troche”. A modern form of troche is the cough drop, so named because it was formed by “dropping” hot, viscous, sugar-based candy onto a sheet or into a mold where it cools to form the troche. Another modern form of troche is the “lozenge”, so named because it was in the shape of a diamond (like on playing cards), which is the meaning of the word “lozenge”. A troche is large enough that a person is able to track where it is in the mouth and move it with their tongue, that is, larger than about 5 mm in at least two dimensions.

U.S. Pat. No. 6,139,861 issued to Mark Friedman surveys methods for adhering a troche to a location within the mouth. These methods include two forms of adherent troches, referred to by Friedman as a “mucoadhesive erodible tablet”. These tablets are formed using polymers such as carboxymethylcellulose, hydroxymethylcellulose, polyacrylic acid, and carbopol-934. An example of a bi-layer tablet is the adhering xylitol troche disclosed in U.S. patent application Ser. No. 11/800,381 (applicant reference 37-3) filed May 4, 2007, which is incorporated herein by this reference. Another form of adherent troche is a flexible device, often called an “oral patch.” Examples include the adherent, soluble oral patch disclosed by the same inventor in U.S. patent application Ser. No. 11/157,054 filed Jun. 20, 2005, which is incorporated herein by this reference, and multi-layer patches, such as those disclosed in PCT patent application serial number PCT/US07/05947 (applicant reference 30-4) by the same inventor entitled Multi-layer medical patch with impermeable center filed Mar. 7, 2007 which is incorporated herein by this reference.

The flexible oral patch mentioned above made by depositing and curing a blob of materials to form a disc with tapered edges that has a maximum thickness in the center less than one-quarter of its diameter. Oral patches made by this process provide a superior mouth feel over adhering tablets made by pressing powders or die cutting a sheet because they are strong enough not to fall apart as they erode, yet they are flexible enough to conform to a surface, and they have thinly tapering edges rather than cliff edges, which combination provides better adhesion (due to flexibility and less chance of catching teeth or tongue on the edge) and better mouth feel. This is particularly true where the desired location for adhering the patch is the roof of the mouth, which is often the preferred location for time releasing ingredients to treat the throat or stomach.

### SUMMARY OF THE INVENTION

In one aspect, the invention is a deposited and cured adhering troche, at least 5 mm in each of at least two dimensions, with a slow rate of dissolution in saliva containing active molecules for topically treating the throat (pharynx), esophagus, or stomach. The troche may include hydrophilic gums that swell when exposed to water, which cause it to dissolve much more slowly than it would otherwise. By their binding to water molecules and swelling, the gum molecules block the flow of water to the active molecules and slow dissolution. The molecules of hydrophilic gums may be one or more of any of cellulose gum, including carboxymethylcellulose, methylcellulose, and hydroxypropylcellulose, any of the other synthetic hydrophilic gums such as carbopol, polyvinyl acid, and polyacrylic acid, any hydrophilic natural vegetable gum such as xanthan gum, konjac gum, tara gum, gellan gum, locust bean gum, acacia gum, alginate, carrageenan, agar, and pectin, or a hydrophilic protein gum such as gelatin or other collagen. The conditions to be treated may be sore throat, peptic ulcers or acid reflux, or reduction of viral replication in the throat and nearby tissues. In an embodiment, the active molecules are one or more of xylitol, Glycyrrhiza extract, antacid, alginate, collagen, bismuth subsalicylate, sucralfate, misoprostol, soluble zinc, such as zinc gluconate, antibiotics such as metronidazole, tetracycline, amoxicillin, or clarithromycin, and anti-virals such as zanamavir, interferon-alpha, or pleconaril.

The dissolution time of the troche in a human mouth is, on average, more than 25 minutes, preferably about an hour. The troche may include hydrophilic gums that swell when exposed to water, which cause it to dissolve much more slowly than it would otherwise. By their binding to water molecules and swelling, the gum molecules block the flow of water to the active molecules and slow dissolution. The molecules of hydrophilic gums may be one or more of any of cellulose gum, including carboxymethylcellulose, methylcellulose, and hydroxypropylcellulose, any of the other synthetic hydrophilic gums such as carbopol, polyvinyl acid, and polyacrylic acid, any hydrophilic natural vegetable gum such as xanthan gum, konjac gum, tara gum, gellan gum, locust bean gum, acacia gum, alginate, carrageenan, agar, and pectin, or a hydrophilic protein gum such as gelatin or other forms of collagen.

In another aspect, the adhering troche may be formed by pressing powders of active molecules and one or more gums into a tablet with a tablet press such that the composition will have lumps of active molecules as large as grains of powder and the molecules of hydrophilic gums are one or more of intermixed within the grains or a coating on the grains or clumped into their own grains as large as grains of powder. The dissolution time of the troche in a human mouth is, on average, more than 25 minutes. The adhesive molecules may comprise acacia gum. Alternatively, they may comprise one or more of gelatin (collagen), alginate, starch, pectin, polyvinylpyrolidone, carboxymethylcellulose, hydroxymethylcellulose, polyvinyl acid, polyacrylic acid, and carbopol. The troche may comprise two layers, a first layer that is not adhesive and a second layer comprised of, by dry weight, at least 30% adhesive molecules.

In another aspect, the invention is a device and method for delivery over time of anti-viral drugs, such as interferon-alpha, or pleconaril, topically to the pharynx and adjoining tissues from an adhering dissolving disc adhered in the mouth. It is particularly suited to delivery of drugs that are not effective when swallowed in a capsule, such as zanamavir (brand name Relenza®). With the invented method and device, the drug disperses through the mucous to adjoining tissues with diminishing concentration in more remote tissues. By delivering a high concentration over time, the concentration is adequate to be effective in all tissues where the virus is likely to make contact and attempt to replicate, while the concentration is far higher than necessary in the mouth and pharynx. Also, some of the drug passes through the mucosal epithelium into the blood and is then distributed to appropriate tissues.

Because concentration of the drug is relatively low in the lungs, the invention is most effective as a preventive before the subject has been exposed to the virus. Once the virus has replicated and spread into the lungs, inhaler delivery is preferred for treatment. For prevention before exposure, the adhering disc is preferred because it is much more pleasant for people to use each day, often two to five times per day, than an inhaler, and the disc avoids complications of irritation of the lungs and airway.

In another aspect, the invention is a method for treating bacterial infections downstream of the mouth (pharynx, esophagus, or stomach) with topically delivered xylitol. The xylitol may be delivered from a troche, including a lollipop or pacifier, an adhering troche, chewing gum, a liquid of high or low viscosity, or grains dissolved in the mouth.

In another aspect, the invention is a method for treating conditions downstream of the mouth (pharynx, esophagus, or stomach) using an adhering dissolving disc to release antacid, alginate, bismuth subsalicylate, soluble zinc, such as zinc gluconate, bioactive vitamin B12 (methylcobalamin), antibiotics such as metronidazole, tetracycline, amoxicillin, or clarithromycin, or anti-virals such as zanamavir, interferon-alpha, or pleconaril.

In another aspect, the invention is a method to treat sore throat with topically applied Glycyrrhiza extract, extracted by any method, including dissolution with any solvent such as water, alcohol, or liquid carbon dioxide, time released from an adhering troche.

In another aspect, the invention is a method to treat sore throat with topically applied collagen time released from an adhering troche.

In another aspect, the invention is a method to treat sore throat with topically applied methylcobalamin.

### BRIEF DESCRIPTION OF THE DRAWINGS

FIG. 1 shows a side view or cross section of a bi-layer adhering troche made with a tablet press.

FIG. 2 shows a side view or cross section of a bi-layer adhering troche made by depositing a blob of paste onto a layer of adhesive material.

### DETAILED DESCRIPTION

The inventor has discovered that adhering troches that time release collagen and Glycyrrhiza extract, made by the methods taught in U.S. Pat. No. 7,201,930 and U.S. patent application Ser. No. 11/157,054, both of which are incorporated by this reference, are effective to combat sore throat and to prevent the throat (pharynx) from becoming significantly sore if applied at the outset of a virus infection where the first symptom is the beginning of a sore throat. To provide adequate levels of collagen to effectively coat the throat, the troche must be at least 25% collagen, such as from gelatin.

Antacids use different combinations of four basic salts—sodium, magnesium, calcium, and aluminum—with hydroxide, carbonate, bicarbonate or similar ions to neutralize acid in the stomach. Antacids, however, can have side effects. Magnesium salt can lead to diarrhea, and aluminum salt may cause constipation. Aluminum and magnesium salts are often combined in a single product to balance these effects. Calcium carbonate antacids, such as Tums, Titralac, and Alka-2, can also be a supplemental source of calcium. They can cause constipation as well. Calcium acetate or calcium lactate can be added to balance undesirable effects. Any of these ingredients can be used to form time release adhering troches. To be adequately effective, the troche must contain at least 200 mg of the antacid compounds.

A troche with large amounts of antacids, anti-bacterials, or anti-virals may be made by mixing molecules to be released with substantial amounts of molecules of a hydrophilic gum that swell when exposed to water. The molecules of hydrophilic gums may be one or more of any of cellulose gum, including carboxymethylcellulose (CMC), methylcellulose, and hydroxypropylcellulose, any of the other synthetic hydrophilic gums such as carbopol, polyvinyl acid, and polyacrylic acid, any hydrophilic natural vegetable gum such as xanthan gum, konjac gum, tara gum, gellan gum, locust bean gum, acacia gum, alginate, carrageenan, agar, and pectin, or a hydrophilic protein gum such as gelatin. For a xylitol troche, low viscosity CMC at about 3.4% is preferred. High viscosity carboxymethylcellulose (CMC 15000 from TIC Gums) required only 2.4% to be effective. A preferred embodiment has 4% low viscosity carboxymethylcellulose (CMC 15 from TIC Gums) and 96% xylitol.

The composition may be formed by pressing powders of molecules to be released and one or more gums into a tablet with a tablet press. Grains of 50 to 350 microns are preferred. The grains may be granulated with a coating of gum on the outside, such as Danisco Xylitab 200 which is granulated with up to but less than 2% carboxymethylcellulose (CMC) as a compression binder. This is not enough CMC to achieve a preferred slow rate of dissolution. Adding at least 1.2% powdered CMC 15 from TIC Gums is effective. Adding 2.1% to 3.5% is preferred, depending on how much CMC is on the xylitol grains as a compression binder and the viscosities of both the CMC on the grains and the added powdered CMC. A 0.7 gram troche of xylitol about 4.5 mm thick dissolved in 47 minutes in the mouth, nearly twice the minimum goal of 25 minutes, with 1.2% added CMC 15. With 2.5% added CMC 15, the dissolution rate was 90 minutes. With 3.5% added CMC 15, the dissolution rate was 120 minutes. A 0.5 gram troche of xylitol with 3.4% low viscosity CMC dissolved in 40-120 minutes, depending on saliva flow.

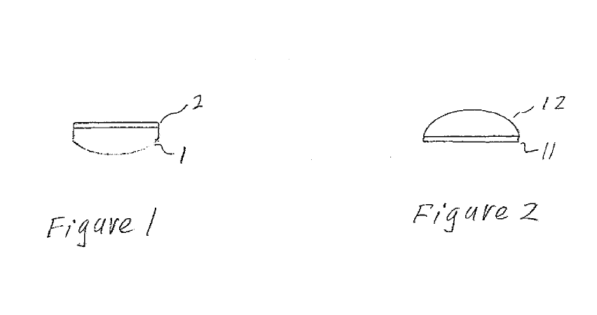

Alternatively, grains of pure material not coated with a gum, such as Danisco Xylitab 300, may be mixed with gum powder and then pressed. Tested and found effective were 3% xanthan plus konjac gum with 0.5% high viscosity CMC, 10% alginate gum, 30% gelatin, 8% alginate with 8% gelatin, 11% acacia gum, 11% pectin, 14% guar gum, and 12% locust bean gum, The composition may be formed into a simple troche as shown in FIGS. 1 and 2

An adhering troche that, when held in a human mouth, erodes, thereby releasing active molecules over time, allows delivery of active molecules without the effect on appearance of chewing or having a candy in one's mouth. It can also be used while sleeping. In a preferred embodiment, the dimensions and structure cause it to take more than 25 minutes to dissolve.

In preferred embodiments, the troches comprise, by dry weight between 50% and 90% solid phase active molecules. Lesser amounts are unattractive to the user who must consequently use more troches. Greater amounts are unachievable because at least 10% is needed for the adhesive and binders that hold it together and slow the release. This leaves between 10% and 50% for the adhesive molecules that adhere in a human mouth as well as binder molecules.

The adhesive molecules may comprise acacia gum. Acacia gum adheres very well to teeth and gingiva, which are the preferred locations for adhesion, and it does not dissolve too fast or leave an unattractive mouth feel. On the surface designed to be adherent, between 80% and 100% acacia gum is preferred for good adhesion. Alternatively, the adhesive molecules may comprise one or more of gelatin, alginate, starch, pectin, polyvinylpyrolidone, carboxymethylcellulose, hydroxymethylcellulose, polyvinyl acid, polyacrylic acid, and carbopol.

The adherent layer can be quite thin. In tests on a preferred size of troche, about 11.5 mm in diameter by 4 to 5 mm thick, the preferred thickness of a layer of about 99% acacia gum was about one-half millimeter. This can be made by bi-layer tablet pressing or by depositing a paste of acacia gum into a mold or by extrusion and die cutting.

The troche can be made as one homogenous composition, such as with highly adhesive molecules like the synthetics, polyvinylpyrolidone, carboxymethylcellulose, hydroxymethylcellulose, polyvinyl acid, polyacrylic acid, and carbopol at about 20 to 50%. Or, it may comprise two layers, a first layer comprised of, by dry weight, at least 75% solid phase active molecules and a second layer comprised of, by dry weight, at least 30% adhesive molecules. To minimize gums required and minimize size for the amount of active delivered, making a bi-layer troche is preferred.

A preferred embodiment of the antacid troche is made on a bi-layer tablet press, putting 85 to 95% of the total weight into an active layer of about 90 to 97% active molecules and 5 to 15% of the weight into an adhesive layer of 30 to 99% adhesive gums. A pressed powder bi-layer round antacid troche, 12 mm in diameter and 4 to 5 mm thick with one-half millimeter of 99% acacia gum in one layer and 3.4% CMC gums in the active as described above adheres well and dissolves in about 40-90 minutes, about double the minimum goal of exceeding 25 minutes.

When making bi-layer tablets with a typical press, a first powder is place in the die, sitting on the lower punch, then the upper punch tamps the powders, leaving the surface having the shape of the upper punch face, then powders of the second layer are added, then an upper punch presses again. A method for making a rounded bi-layer oral adhesive tablet on a typical bi-layer press is to configure the press to have a lower punch that is dish shaped to produce a rounded tablet surface and an upper punch that is substantially flat. One makes tablets with the press by first pouring into the die a granular material that is not intended to be oral adhesive, then tamping the granular material with the upper punch, then adding to the die oral adhesive granular material, then compressing the granular materials between the two punches to form a tablet that is substantially flat on an oral adhesive side and rounded on the other side.

The dish shape may be approximately a portion of a sphere. The dish shape may be produced by a face on a lower punch that is substantially flat in a center area and the center area is surrounded by a raised edge which forms a dish shape. For a troche 12 mm in diameter, a suitable amount of dish is 1.5 to 3 mm, preferably 2.1 mm, with a total tablet thickness of 4 to 5 mm.

The compositions and troches described above may be used for combating bacterial effects downstream of the mouth. Dissolving troches comprising crystalline xylitol which, when exposed to saliva in a human mouth, on average, release xylitol molecules more slowly than a troche of pure xylitol are supplied to consumers. The consumers are instructed to place a troche in their mouths and keep it there until the xylitol in the troche is dissolved. In one embodiment, the dissolution time of the troches in a human mouth is, on average, more than 25 minutes. The greater the number of hours each day with a troche releasing xylitol in the mouth, the better, up to a point of diminishing returns. Using one troche as described above at the end of each day and one after each meal, at least four per day, which adds up to two or more hours per day, is presently preferred.

The bacterial effects combated include bacterial pharyngitis, esophogitis, and bacterial ulcers. Users adhere a troche to a tooth or adjoining gums in the rear of their mouths at any time of day or night, preferably after each meal or snack, at least four times per day. Placing it on the tongue side of the teeth causes it to erode more quickly than placing it on the cheek side. It can instead be adhered to the cheek.

Zanamavir is well suited to delivery by the invented device and method. It is not metabolized by the body and is excreted in the urine without change. It is presently delivered topically by dry powder inhaler (approximately 4% to 17% of the inhaled dose is systemically absorbed) and is used mainly as a treatment, not a preventive. Rare side effects have been found other than those caused by the delivery vehicle. When delivered to the stomach such as by capsule, 2% is absorbed into the blood. When time released into the mouth, absorption through the mouth and throat lining may be higher, closer to the 4-17% absorption through the lungs.

In a preferred embodiment, the device is adjusted to time release the drug over a median time of about 60 minutes, up to 120 minutes for people with low saliva flow and down to 30 minutes for people with exceptionally high saliva flow. Patients are instructed not to eat or drink while the disc is releasing the drug.

The recommended preventive use of zanamavir delivered by the invented device and method is for people to place an adhering time release disc in their mouths about 30-60 minutes before they enter a room with people who might be infectious, up to twice a day. Most people would therefore use the product during flu season each morning five days per week and before they visit their grandchildren or a congested public interior space. Also, it would be used by family members when one person becomes ill with flu-like symptoms, taken twice a day or, if the family spends mornings apart, taken just before the family convenes in the evening. A person who is ill might take it two or three times a day for treatment.

The anti-viral troche may be formed by pressing powders of zanamavir or an anti-picornavirus drug and one or more gums into a tablet with a tablet press. Zanamavir grains of 20 to 200 microns are preferred. The grains may be granulated with a coating of gum on the outside with 2-10% carboxymethylcellulose (CMC) as a compression binder.

Alternatively, grains of pure zanamavir or an anti-picornavirus drug may be mixed with gum powder and then pressed. Tested and found effective were 3% xanthan plus konjac gum with 0.5% high viscosity CMC, 10% alginate gum, 30% gelatin, 8% alginate with 8% gelatin, 11% acacia gum, 11% pectin, 14% guar gum, and 12% locust bean gum,

The composition may be formed into a simple troche as shown in FIGS. 1 and 2, a troche with a handle to form a lollipop or a troche in the form of a child's pacifier. Such a lollipop or pacifier may be used by a child younger than six without risk of aspiration of the troche. A suitable manufacturing method is the common method of making of lollipops by heating with kneading to a hot, slowly flowable paste, then forming onto the stick with use of molds, then cooling.

The embodiment of an adherent troche that, when held in a human mouth, erodes, thereby releasing zanamavir or an anti-picornavirus drug over time, allows delivery of anti-viral molecules without the effect on appearance of chewing or having a mint in one's mouth. It can also be used while sleeping. In a preferred embodiment, the dimensions and structure cause it to take more than 25 minutes to dissolve.

The preferred embodiment of the troche is made on a bi-layer tablet press, putting 60 to 80% of the total weight into a zanamavir layer with 10-20 mg zanamavir plus excipients and 20 to 40% of the weight into an adhesive layer of 30 to 99% adhesive gums. A pressed powder bi-layer round zanamavir troche, 8 mm in diameter and 2 to 4 mm thick with one-half millimeter of 99% acacia gum in one layer and CMC gums in the zanamavir as described above adheres well and dissolves in about 50-120 minutes.

While particular embodiments of the invention have been described above the scope of the invention should not be limited by the above descriptions but rather limited only by the following claims.

What is claimed:

1-14. (canceled)

15. An adhering troche, at least 5 mm in each of at least two dimensions, with a slow rate of dissolution in saliva comprising at least 200 mg of antacid compounds.

16. The troche of claim 15 wherein, when exposed to saliva in a human mouth, the dissolution time of the troche is more than 25 minutes at typical mid day levels of saliva flow.

17. The troche of claim 15 wherein the antacid compounds comprise at least one of: sodium bicarbonate, calcium bicarbonate, magnesium bicarbonate, aluminum bicarbonate, calcium carbonate, magnesium carbonate, aluminum carbonate, sodium carbonate, magnesium trisilicate, alumina, magnesium oxide, magnesium hydroxide, and aluminum hydroxide.

18-31. (canceled)

32. The troche of claim 15 comprising two layers, a first adhesive layer of 30 to 99% adhesive gums that adheres in a human mouth and a second delivery layer that contains the antacid compounds.)

33. The troche of claim 32 wherein the second delivery layer that contains the antacid compounds comprises at least 75% antacid compounds.)

33. The troche of claim 32 having a total thickness of about 4 to 5 millimeters.
